(19) United States
(12) Patent Application Publication
(10) Pub. No.: US 2021/0379581 A1
(43) Pub. Date: Dec. 9, 2021

(54) Magnetic PCR Assay And Uses Thereof

(71) Applicant: Western Digital Technologies, Inc., San Jose, CA (US)

(72) Inventor: Daniel BEDAU, San Jose, CA (US)

(21) Appl. No.: 16/894,481

(22) Filed: Jun. 5, 2020

(51) Int. Cl. B01L 3/00; C12Q 1/686; C12N 15/10

(52) U.S. Cl. CPC B01L 3/5027; C12Q 1/686; B01L 2300/0867; B01L 2300/0627; B01L 2300/18; C12N 15/1096

(57) ABSTRACT

Embodiments of the present disclosure generally relate to diagnostic apparatus and uses thereof. The apparatus and methods described herein can be useful for, e.g., rapid detection of SARS- and SARS-related coronaviruses. In an embodiment is provided an apparatus for detecting a nucleotide sequence that includes a fluidic channel coupled to one or more reservoirs, the fluidic channel comprising a sample introduction component, a polymerase chain reaction component, and a detection component. The apparatus further includes a temperature control device to heat or cool the fluidic channel, one or more sensors to detect the presence or absence of a magnetic nanoparticle, and a processor coupled to the one or more sensors and to the temperature control device. In another embodiment is provided a method for detecting an analyte that includes introducing a sample to an apparatus described herein, performing one or more processes to the sample, and detecting the analyte.

### BACKGROUND OF THE DISCLOSURE

### Field of the Disclosure

Embodiments of the present disclosure generally relate to diagnostic apparatus and uses thereof. Specifically, embodiments of the present disclosure relate to magnetic polymerase chain reaction (PCR) assays and uses thereof.

### Description of the Related Art

Assays to determine the presence of components in a sample are important diagnostic tools. Frequently, however, the components of interest within a sample, e.g., a nucleic acid, a virus, a bacterium, a protein, are minor constituents of the sample and can therefore be challenging to detect.

Conventional methods of amplifying and detecting, e.g., nucleic acids typically involve PCR assays with fluorescence detection. Although fluorescence-based detection methods can provide accurate diagnostic information, the instrumentation used—including the large optical detection apparatus, UV light sources, and fluorescent probes—is expensive and fabrication of such instrumentation does not provide commercially viable scale-up. Such complications and high production costs render fluorescent-based apparatus uneconomical and unsuitable for high-volume manufacturing. Moreover, the large instrumentation typically employed for fluorescence-based apparatus is not portable for on-site diagnoses.

There is a need for improved apparatus and methods for detecting components of interest within a sample that overcome one or more deficiencies of conventional apparatus and methods.

### SUMMARY OF THE DISCLOSURE

Embodiments of the present disclosure generally relate to diagnostic apparatus and uses thereof. Specifically, embodiments of the present disclosure relate to magnetic polymerase chain reaction (PCR) assays and uses thereof. The apparatus and methods described herein can allow for in-situ, real-time, and/or rapid diagnosis of, e.g., a virus such as SARS- and SARS-related coronaviruses.

In an embodiment is provided an apparatus for detecting a nucleotide sequence that includes a fluidic channel coupled to one or more reservoirs, the fluidic channel comprising a sample introduction component, a polymerase chain reaction component, and a detection component. The apparatus further includes a temperature control device to heat or cool the fluidic channel, one or more sensors to detect the presence or absence of a magnetic nanoparticle, and a processor coupled to the one or more sensors and to the temperature control device.

In another embodiment is provided an apparatus for detecting a nucleotide sequence that includes a fluidic channel coupled to one or more reservoirs, the fluidic channel comprising a sample introduction component, a polymerase chain reaction component, and a detection component. The apparatus further includes a temperature control device to heat or cool the fluidic channel. The apparatus further includes one or more sensors to detect the presence or absence of a magnetic nanoparticle, the one or more sensors adjacent to the temperature control device. The apparatus further includes a processor coupled to the one or more sensors and to the temperature control device,

In another embodiment is provided a method for detecting an analyte that includes introducing a sample to an apparatus, the apparatus comprising a temperature control device configured to perform a polymerase chain reaction operation and one or more sensors to detect the presence or absence of a magnetic nanoparticle. The method further comprises performing one or more processes to the sample, and detecting the analyte.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1A is a top view of an example apparatus for detecting an analyte according to at least one embodiment of the present disclosure.

FIG. 1B is a top view of an example apparatus for detecting an analyte according to at least one embodiment of the present disclosure.

FIG. 2 is a top view of an example apparatus for detecting an analyte according to at least one embodiment of the present disclosure.

FIG. 3 is an example magnetic nanoparticle (MNP) according to at least one embodiment of the present disclosure.

FIG. 3A is an example detection area of an example apparatus for detecting an analyte according to at least one embodiment of the present disclosure.

FIG. 3B is an example detection area of an example apparatus for detecting an analyte according to at least one embodiment of the present disclosure.

FIG. 4A is an example detection area of an example apparatus for detecting an analyte according to at least one embodiment of the present disclosure.

FIG. 4B is an example detection area of an example apparatus for detecting an analyte according to at least one embodiment of the present disclosure.

FIG. 5 is an example readout signal according to at least one embodiment of the present disclosure.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Embodiments of the present disclosure generally relate to diagnostic apparatus and uses thereof. Specifically, embodiments of the present disclosure relate to magnetic polymerase chain reaction (PCR) assays and uses thereof. The inventor has discovered improved apparatus and methods for detecting components of interests (analytes) that overcome deficiencies of conventional diagnostic tools and methods. The apparatus and methods provided herein address major deficiencies of current diagnostic tools and methods by being able to analyze small amounts of analytes, e.g., DNA/RNA, reduce analysis time, lower costs (e.g., elimination of expensive hardware and optics, less people involved in diagnoses, reduced procedures and time), eliminate fluid transfers which cause contamination, among others.

In contrast to conventional diagnostic apparatus, apparatus of the present disclosure are low cost and portable so as to enable, at least, on-site diagnoses. Unlike fluorescence-based apparatus which use expensive optical systems, and hardware, apparatus of the present disclosure can tolerate easy scale-up with much lower production costs. The magnetic-based detection apparatus can use very little hardware, e.g., a semiconductor chip, rendering the improved detection apparatus much smaller and more portable than fluorescence-based detection apparatus. Fluorescence-based detection apparatus typically employ 96-well trays making them unsuitable for lab-on-a-chip diagnostic tools. In contrast, the magnetic-based detection apparatus of the present disclosure can be employed in a lab-on-a-chip device. The number of wells used for fluorescence-based apparatus can also potentially lead to contamination, a problem not seen with various embodiments provided herein.

Moreover, the magnetic sensors can be fabricated using modified semiconductor manufacturing technologies such that tens of thousands of sensors can be made on a single wafer. Optics-based detection apparatus, such as fluorescence-based detection apparatus, cannot be made so simply and efficiently. Accordingly, the magnetic-based detection apparatus can be relatively cheap to manufacture in large quantities. As a result, the apparatus of the present disclosure have greatly improved commercial scale-up than conventional optics-based detection apparatus.

The apparatus and methods described herein can allow for in-situ, real-time, and/or rapid diagnosis. The apparatus can be a single device and/or a disposable device. Generally, the apparatus can perform extraction of a sample, PCR amplification, and magnetic detection of an analyte of interest. As a non-limiting example, and with respect to extraction, the device can extract a biological material, e.g., DNA and/or RNA, from a sample (such blood, saliva, etc.) for analysis.

Although some of the disclosure herein is provided in the context of nucleic acid detection, it is to be understood that the embodiments herein generally can be used to detect any type of molecule or analyte to which a magnetic particle (e.g., a magnetic nanoparticle) can be attached. The disclosure presumes that the particles attached are magnetic nanoparticles, but this presumption is exemplary and is not intended to be limiting. Any molecule type that can be labeled by a magnetic nanoparticle can be detected using the methods and detection devices disclosed herein. Such molecule types can be biologic molecule types, such as proteins, antibodies, etc. For example, the disclosures herein can be used to detect nucleic acids. The disclosures herein may also be used to detect non-biologic (inorganic or non-living) molecules, such as contaminants, minerals, chemical compounds, etc. The presentation of portions of the disclosure in the context of nucleic acid detection is not intended to limit the scope of the present disclosure.

In addition, although some of the disclosure is provided in the context of RNA viruses, the devices and apparatus can be useful for DNA viruses. For example, for DNA viruses, the reverse transcription operation can be removed from, or “turned off” in apparatus and methods described herein. Sample preparation, such as pretreatment operation(s) and extraction operations may also be different.

In some embodiments, the apparatus and methods described herein allow for in-situ, real-time diagnostic of, e.g., a biological material such as a virus. Non-limiting examples of viruses that can be detected by apparatus, e.g., assays, described herein can include HCoV-HKU1, HCoV-OC43, HCoV-NL63, HCoV-229E, MERS-CoV, Influenza A (H1N1/09), Influenza A (H3N2), Influenza A (H5N1), Influenza B, Rhinovirus/Enterovirus, Respiratory syncytial virus (A/B), Parainfluenza 1 virus, Parainfluenza 2 virus, Parainfluenza 3 virus, Parainfluenza A and B virus, Human metapneumovirus, Adenovirus, Human Bocavirus, Legionella spp., Mycoplasma spp., and a combination thereof. For time sensitive analysis, as in viral infection diagnoses, fast analysis rates can be important. The apparatus described can complete an analysis much faster than conventional methods. There is no need to, e.g., transfer the cells to different tubes and ship the cells to a different location for analysis. In addition, the apparatus and methods described herein can be performed on small sample volumes.

### Magnetic PCR Assay

The present disclosure generally relates to magnetic PCR assay, such as a magnetic PCR assay. These magnetic PCR assays can be useful for, e.g., the rapid detection of a virus such as SARS- and SARS-related coronaviruses. The magnetic PCR assay may be in the form of a microfluidic device such as a lab-on-a-chip device. The magnetic PCR Assay device described herein can produce results faster while consuming fewer amounts of reagents and generating less wastes and hazardous materials. It is low cost, highly portable, and easy to operate. Direct current (DC) and alternating current (AC) power can be used to control the behavior and properties of samples and analytes (e.g. cells, bacteria, virus, proteins, nucleotides, etc.) in the fluidic channels. For example, AC and/or DC power can provide localized heating, fluid mixing, and bio-particle handling such as cell sorting, trapping and positioning, cell stretching, and lysing.

In some embodiments, the apparatus performs one or more of the following operations for RNA virus detection: pretreatment to lyse the cell; RNA extraction to remove RNA from the cellular material; reverse transcription of the RNA to a complementary DNA; PCR to amplify the DNA; and detection of the DNA. For DNA virus detection, and in some embodiments, reverse transcription can be “turned off” in apparatus and RNA extraction can be replaced by DNA extraction from the cellular material. Generally, a sample comprising a component of interest (e.g., an analyte) can be introduced to a fluidic channel of the apparatus. As the sample moves through the fluidic channel of the apparatus, one or more of the aforementioned operations are performed on the analyte. Detection of the analyte can provide diagnostic information to the user.

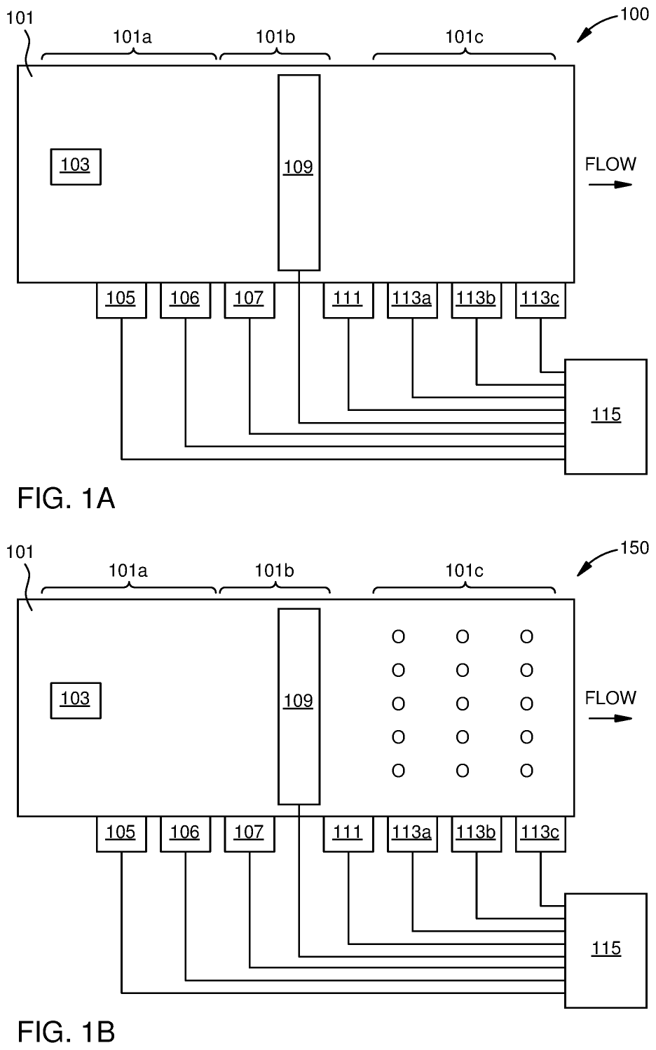

FIG. 1A is a top view of an example apparatus 100 for detecting an analyte, e.g., DNA and/or RNA of a virus, according to at least one embodiment of the present disclosure. The example apparatus 100 can be a magnetic PCR assay. In some embodiments, the example apparatus 100 can be a lab-on-a-chip apparatus.

The example apparatus 100 can include a fluidic channel 101. In at least one embodiment, the fluidic channel 101 can have a diameter of micrometers (μm) to millimeters, such as at least about 10 μm, such as from about 10 μm to about 2 mm, such as from about 50 μm to about 1 mm or about 10 μm to about 1 mm. The fluidic channel 101 can be segregated into at least three components—a sample introduction area 101a, a polymerase chain reaction area (PCR area) 101b, and a detection area 101c. The sample introduction area 101a can include an opening 103 where the sample is introduced. The opening 103 can be coupled to the fluidic channel 101. In some embodiments, the opening 103 can range from a few micrometers in diameter to a few millimeters in diameter depending on the application.

The fluidic channel 101 can be coupled to one or more reservoirs. The reservoirs can hold components and/or reaction mixture precursors that mix and/or interact (e.g., chemically and/or physically) with the sample as the sample moves through the fluidic channel 101. Such reaction mixture precursors can include chemicals, e.g., solvents, reagents, catalysts, etc., that upon interacting with the sample, form a reaction mixture and transform the sample. That sample can include cells and cellular material. A first reservoir 105 of the one or more reservoirs can include components and/or reaction mixture precursors that, upon interaction with the sample, lyse the cell and/or condition the sample for PCR. A second reservoir 107 of the one or more reservoirs can include PCR components and/or reaction mixture precursors that, upon interaction with the sample, amplify DNA. A third reservoir 111 of the one or more reservoirs can include components and/or reaction mixture precursors that, upon interaction with the sample, react a nucleotide sequence with a functionalized magnetic nanoparticle (MNP).

The first reservoir 105, the second reservoir 107, and the third reservoir 111 can each, independently, include one or more reservoirs. For example, first reservoir 105 can include reservoir 105a and reservoir 105b (not shown). Reservoir 105a can include components and/or reaction mixture precursors that, upon interaction with the sample, perform a pretreatment operation that break open (or lyse) the cell of the sample. Reservoir 105b can include components and/or reaction mixture precursors that, upon interaction with the sample, extract the analyte of interest from the other cellular material following cell lysis.

A fourth reservoir 106 can be used in the example apparatus 100 when it is desired to convert RNA to complementary DNA or cDNA. Accordingly, the fourth reservoir 106 can include components and/or reaction mixture precursors that, upon interaction with the sample, perform a reverse transcription reaction on the RNA. Components and/or reactions mixtures that are held in the reservoirs, such as those components and/or reaction mixture precursors to lyse the cell, to extract the RNA, to perform reverse transcription, to perform PCR, to attach MNPs to nucleotides, and to condition the sample for PCR can be commercially obtained. Probes and primers, discussed below, can also be purchased.

The fluidic channel 101 can be coupled to a temperature control device 109. Coupling of the fluidic channel 101 to the temperature control device 109 can take multiple forms. For example, the fluidic channel 101 can be coupled to the temperature control device 109 as shown in FIG. 1A. As another example, the example apparatus 100 can be placed on a hot plate (a temperature control device) and heated. Here, the hot plate can be located in a stand-alone machine outside of the example apparatus 100, and as such, the fluidic channel 101 is mechanically coupled to the temperature control device 109. As another example, the example apparatus 100 can be placed under an infrared (IR) lamp (a temperature control device) and heated by the IR lamp. Here, the IR lamp can be located in a stand-alone machine outside of the example apparatus 100, and as such, the fluidic channel 101 is optically coupled to the temperature control device 109.

The temperature control device 109 (e.g., a heater) can be configured to perform a polymerase chain reaction operation as discussed below. Briefly, the temperature control device can heat and/or cool down the nucleotide during one or more phases of the PCR reaction. In some embodiments, the temperature control device 109 can include a sensor to measure the temperature applied to the fluid during, e.g., PCR. The temperature control device itself could serve as a sensor due to a resistance change with temperature. In these and other embodiments, the temperature control device can be a serpentine coil heater and the sensor can be placed in a gap between the heater coils and/or to the side of the heater coils. In some embodiments, a closed-loop feed-back system can be applied where the heater can be controlled by a sensor.

The temperature control device 109 (e.g., a heater) can be configured to perform a polymerase chain reaction operation as discussed below. Briefly, the temperature control device can heat and/or cool down a nucleotide sequence during one or more phases of the PCR reaction.

The fluidic channel 101 can be coupled to one or more magnetic sensors 113. The magnetic sensors 113 can each, independently, include a functionalized surface “probe” that allows detection of an analyte. Although the magnetic sensors 113 are shown on the side of the fluidic channel 101, the magnetic sensors 113 (shown as “O”) can be located in detection area 101c, as shown in FIG. 1B. In such a design, much of the fluid can come into contact with the magnetic sensors. Other elements of FIG. 1B can be the same as that described in relation to FIG. 1A. The probe can be chemically attached to a part of the magnetic sensor 113, e.g., chemically attached on or near a magnetic sensor 113. The probes can be an oligonucleotide having a specific or desired nucleotide sequence that is complementary to a particular sequence on one of the strands of a DNA duplexes flowing through the fluidic channel 101. As described below, these DNA duplexes are tagged with a MNP to form MNP-tagged nucleotides. When the MNP-tagged nucleotide interacts with probe, the MNP is brought close to one of the magnetic sensors 113 and a determination of the nucleotide present can be made.

In some embodiments, the one or more magnetic sensors 113 can include at least one magnetic sensor 113a having a probe that is specific for a particular analyte, e.g., a particular nucleotide. In at least one embodiment, the one or more magnetic sensors 113 can include at least one magnetic sensor 113b having a probe that is specific for a different analyte, e.g., a particular nucleotide. For example, magnetic sensor 113a can include a probe specific for Wuhan-CoV and will not detect SARS-CoV. The magnetic sensor 113b can include a probe specific for SARS-CoV and will not detect Wuhan-CoV. In some embodiments, at least one of the one or more magnetic sensors 113, e.g., magnetic sensor 113c, can include a probe that binds non-specific analytes to ensure that the test was run.

In such embodiments, a multiplicity of viruses (two or more) can be detected in a single run using two or more magnetic sensors 113. Alternatively, the example apparatus 100 can contain only one magnetic sensor (not including the magnetic sensor 113c that ensures the test was run) which detects a specific type of virus.

Various temperature controlled zones can be added to the example apparatus 100 to heat and/or cool the sample (with or without fluid) flowing through the fluidic channel 101 at various stages of the example apparatus 100 and/or to stimulate movement of the sample/fluid through the fluidic channel 101. Such temperature controlled zones (having, e.g., heating and cooling elements) can also be placed along various parts of the fluidic channel to aid in various operations such as pre-treatment of the sample, extraction, reverse transcription, amplification, and detection. Temperatures suitable to promote such operations are known in the art. The temperature controlled zones can correspond to the sample introduction area 101a, the PCR area 101b, and the detection area 101c. During fabrication of the apparatus, the temperature controlled zones can be fabricated as a thin metal film/wire patterned onto the surface of a layer of the apparatus.

The sample and/or analyte (which can be in the form of a solid, liquid, gas, and/or plasma) can be diluted by, e.g., fluids flowing out of one or more reservoirs coupled to the fluidic channel. Accordingly, and in some embodiments, various fluids can enter the fluidic channel to aid in one or more operations. The fluids can be released from the reservoirs mentioned above (e.g., reservoir 105a, reservoir 105b, second reservoir 107, third reservoir 111, and/or fourth reservoir 106) or other reservoirs not shown in FIG. 1. Such fluids can include, but are not limited to, ethanol, isopropanol, methanol, amyl alcohol, water, urea, guanidinium thiocyanate, HCl (20%), buffer solutions, or a combination thereof.

Such fluids can aid in movement of the sample. Movement of the sample and/or analyte in the fluidic channel 101 from the opening 103 to the magnetic sensors 113 (in the direction of the arrow) can be controlled by, e.g., capillary action, temperature, a pumping mechanism (e.g., a piezoelectric pump), electrodes, and the like. Such elements can be placed along various parts of the fluidic channel. For example, the fluid can be pumped by heating the sample and creating a bubble. The bubble can then act to push the sample towards the detection area. Additionally, or alternatively, first reservoir 105, second reservoir 107, third reservoir 111, and/or fourth reservoir 106 can be at least partially covered with a membrane. Applying a force on the membrane can then act to move the sample towards the detection area 101c. Accordingly, the analyte(s) in the fluid move from the sample introduction area 101a on the proximal end to the detection area 101c on the distal end. Movement of the components and/or reaction mixture precursors from various reservoirs, e.g., first reservoir 105, second reservoir 107, third reservoir 111, and/or fourth reservoir 106 and into the fluidic channel 101 can be controlled in a similar manner such that the components and/or reaction mixture precursors can interact with the sample and/or analyte.

Elements that sense movement of the sample and/or analyte as the sample fluid flows through the fluidic channel can be added to the example apparatus 100 such as a piezoelectric or piezoresistive element to sense movement. These elements can be added to apparatus on one or both sides of the fluidic channel to sense up and down movement as well as left and right movement. Additionally, or alternatively, a sensing element could also be used to measure the elasticity (e.g., stiffness) of a sample like a cell to determine how hard or how soft it is. In some embodiments, the example apparatus 100 can include one or more chambers for mixing a sample with a reagent, e.g., a dried reagent and/or a liquid reagent.

The example apparatus 100 can further include one or more processors 115 coupled to, e.g., the one or more magnetic sensors 113, the temperature control device 109, and/or first reservoir 105, second reservoir 107, third reservoir 111, and/or fourth reservoir 106. The one or more processors 115 can be coupled to other elements of the example apparatus 100 such as the opening 103, temperature controlled zones, etc. In at least one embodiment, the one or more processors 115 is an external control circuit connected to the example apparatus 100.

In some embodiments, pumping elements can be added to the example apparatus 100 in order to draw the sample and/or analyte to the magnetic sensors. Pumping can be achieved with a simple syringe operated manually, or with a micro-pump (e.g., a small micro-machined pump of dimension comparable to the analysis unit), a membrane pump, or with any other type of pumping system capable of producing sufficient suction to draw the sample and/or analyte in the example apparatus 100. Such pumping elements can be located at various positions along the fluidic channel 101, such as in the sample introduction area 101a, the PCR area 101b, and the detection area 101c. For example, pumping elements can be placed between the sample introduction area 101a and the PCR area 101b to pump the sample from the opening 103 to the PCR area 101b. The example apparatus 100 can be powered by any means including but not limited to batteries, AC power supply, DC power supply, and the like.

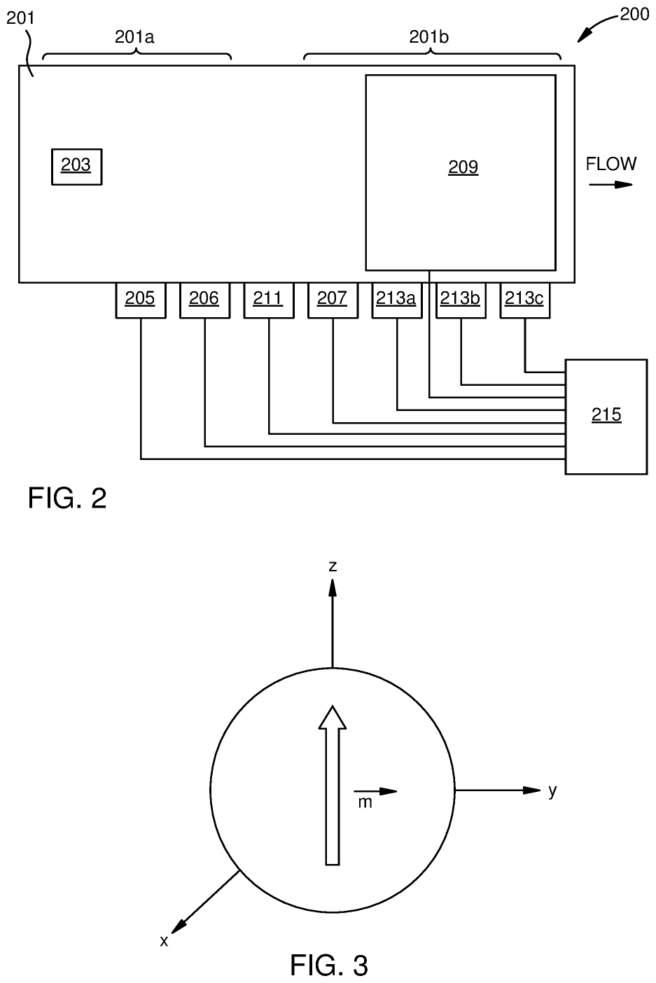

FIG. 2 is a top view of an example apparatus 200 for detecting an analyte, e.g., DNA and/or RNA of a virus, according to at least one embodiment of the present disclosure. In some embodiments, the example apparatus 200 can be a lab-on-a-chip apparatus. At least one feature of example apparatus 200 that is different from example apparatus 100 is the PCR area and detection area being combined in a single area as 201b. Accordingly, example apparatus 200 can have the PCR area and detection area adjacent to one another, between one another, close by one another, next to one another, and/or enmeshed with one another. In some embodiments, the PCR area and detection area can be coplanar.

Combining the PCR area and the detection area can allow real-time detection of a nucleotide as the nucleotide is amplified. In addition, for example apparatus 200, the reservoir that contains components and/or reaction mixture precursors that, upon interaction with the sample, react a nucleotide sequence with a functionalized MNP (e.g., third reservoir 211), is located along the flow path of the fluidic channel prior to the reservoir that contains PCR components and/or reaction mixture precursors to amplify DNA (e.g., second reservoir 207).

The example apparatus 200 can include a fluidic channel 201. In at least one embodiment, the fluidic channel 101 can have a diameter of micrometers (μm) to millimeters, such as at least about 10 μm, such as from about 10 μm to about 2 mm, such as from about 50 μm to about 1 mm or about 10 μm to about 1 mm. The example apparatus 200 can be a magnetic PCR assay. The fluidic channel 101 can be segregated into at least two components—a sample introduction area 201a and a PCR/detection area 201b. The sample introduction area 201a can include an opening 203 where the sample is introduced. The opening 203 can be coupled to the fluidic channel 201. In some embodiments, the opening 203 can range from a few micrometers in diameter to a few millimeters in diameter depending on the application.

The fluidic channel 201 can be coupled to one or more reservoirs. The one or more reservoirs can hold components and/or reaction mixture precursors that mix and/or interact (e.g., chemically and/or physically) with the sample as the sample moves through the fluidic channel 201. Such reaction mixture precursors can include chemicals, e.g., solvents, reagents, catalysts, etc., that upon interacting with the sample, form a reaction mixture and transform the sample. That sample can include cells and cellular material. A first reservoir 205 of the one or more reservoirs can include components and/or reaction mixture precursors that, upon interaction with the sample, lyse the cell and/or condition the sample for PCR. A second reservoir 207 of the one or more reservoirs can include PCR components and/or reaction mixture precursors that, upon interaction with the sample, amplify DNA. A third reservoir 211 of the one or more reservoirs can include components and/or reaction mixture precursors that, upon interaction with the sample, react a nucleotide sequence with a functionalized magnetic nanoparticle (MNP).

The first reservoir 205, the second reservoir 207, and the third reservoir 211 can each, independently, include one or more reservoirs. For example, first reservoir 205 can include reservoir 205a and reservoir 205b (not shown). Reservoir 205a can include components and/or reaction mixture precursors that, upon interaction with the sample, perform a pretreatment operation that break open (or lyse) the cell of the sample. Reservoir 205b can include components and/or reaction mixture precursors that, upon interaction with the sample, extract the analyte of interest from the other cellular material following cell lysis.

A fourth reservoir 206 can be used in the example apparatus 200 when it is desired to convert RNA to complementary DNA or cDNA. Accordingly, the fourth reservoir 206 can include components and/or reaction mixture precursors that, upon interaction with the sample, perform a reverse transcription reaction on the RNA. Components and/or reactions mixtures that are held in the reservoirs, such as those components and/or reaction mixture precursors to lyse the cell, to extract the RNA, to perform reverse transcription, to perform PCR, to attach MNPs to nucleotides, and to condition the sample for PCR can be commercially obtained. Probes and primers, discussed below, can also be purchased.

The fluidic channel 201 can be coupled to a temperature control device 209. Coupling of the fluidic channel 201 to the temperature control device 209 can take multiple forms. For example, the fluidic channel 201 can be coupled to the temperature control device 209 as shown in FIG. 2. As another example, the example apparatus 200 can be placed on a hot plate (a temperature control device) and heated. Here, the hot plate can be located in a stand-alone machine outside of the example apparatus 200, and as such, the fluidic channel 201 is mechanically coupled to the temperature control device 209. As another example, the example apparatus 200 can be placed under an infrared (IR) lamp (a temperature control device) and heated by the IR lamp. Here, the IR lamp can be located in a stand-alone machine outside of the example apparatus 200, and as such, the fluidic channel 201 is optically coupled to the temperature control device 209.

The temperature control device 209 (e.g., a heater) can be configured to perform a polymerase chain reaction operation as discussed below. Briefly, the temperature control device can heat and/or cool down the nucleotide during one or more phases of the PCR reaction.

The fluidic channel 201 can be coupled to one or more magnetic sensors 213. The magnetic sensors 213 can each, independently, include a probe that allows detection of an analyte. Although the magnetic sensors 213 are shown on the side of the fluidic channel 201, the magnetic sensors 213 can be located in PCR/detection area 201b, similar to that as shown in FIG. 1B. In such a design, much of the fluid can come into contact with the magnetic sensors. As described above, the PCR area and the detection area are combined in one area as PCR/detection area 201b. In such embodiments, a heating wire can meander around the various magnetic sensors 213 to control the temperature of the sample during the PCR reaction.

The probe can be chemically attached to a part of the magnetic sensor 213, e.g., chemically attached on or near a magnetic sensor 213. The probes can be an oligonucleotide having a specific or desired nucleotide sequence that is complementary to a particular sequence on one of the strands of a DNA duplexes flowing through the fluidic channel 201. As described below, these DNA duplexes are tagged with a MNP to form MNP-tagged nucleotides. When the MNP-tagged nucleotide interacts with probe, the MNP is brought close to one of the magnetic sensors 213 and a determination of the nucleotide present can be made.

In some embodiments, the one or more magnetic sensors 213 can include at least one magnetic sensor 213a having a probe that is specific for a particular analyte, e.g., a particular nucleotide. In at least one embodiment, the one or more magnetic sensors 213 can include at least one magnetic sensor 213b having a probe that is specific for a different analyte, e.g., a particular nucleotide. For example, magnetic sensor 213a can include a probe specific for Wuhan-CoV and will not detect SARS-CoV. The magnetic sensor 213b can include a probe specific for SARS-CoV and will not detect Wuhan-CoV. In some embodiments, at least one of the one or more magnetic sensors 213, e.g., magnetic sensor 213c, can include a probe that binds non-specific analytes to ensure that the test was run.

In such embodiments, a multiplicity of viruses (two or more) can be detected in a single run using two or more magnetic sensors 213. Alternatively, the example apparatus 200 can contain only one magnetic sensor (not including the magnetic sensor 213c that ensures the test was run) which detects a specific type of virus.

Various temperature controlled zones can be added to the example apparatus 200 to heat and/or cool the sample (with or without fluid) flowing through the fluidic channel 201 at various stages of the example apparatus 200 and/or to stimulate movement of the sample/fluid through the fluidic channel 201. Such temperature controlled zones (having, e.g., heating and cooling elements) can also be placed along various parts of the fluidic channel to aid in various operations such as pre-treatment of the sample, extraction, reverse transcription, amplification, and detection. Temperatures suitable to promote such operations are known in the art. The temperature controlled zones can correspond to the sample introduction area 201a and the PCR/detection area 201b.

The sample and/or analyte (which can be in the form of a solid, liquid, gas, and/or plasma) can be diluted by, e.g., fluids flowing out of one or more reservoirs coupled to the fluidic channel. Accordingly, and in some embodiments, various fluids can enter the fluidic channel to aid in one or more operations. The fluids can be released from the reservoirs mentioned above (e.g., reservoir 205a, reservoir 205b, second reservoir 207, third reservoir 211, and/or fourth reservoir 206) or other reservoirs not shown in FIG. 2. Such fluids can include ethanol, isopropanol, methanol, amyl alcohol, water, urea, guanidinium thiocyanate, HCl (20%), buffer solutions, or a combination thereof.

Such fluids can aid in movement of the sample. Movement of the sample and/or analyte in the fluidic channel 201 from the opening 203 to the magnetic sensors 213 (in the direction of the arrow) can be controlled by, e.g., capillary action, temperature, a pumping mechanism (e.g., a piezoelectric pump), electrodes, and the like. For example, the fluid can be pumped by heating the sample and creating a bubble. The bubble can then act to push the sample towards the detection area. Additionally, or alternatively, first reservoir 205, second reservoir 207, third reservoir 211, and/or fourth reservoir 206 can be at least partially covered with a membrane. Applying a force on the membrane can then act to move the sample towards the PCR/detection area 201b. Such elements can be placed along various parts of the fluidic channel. Accordingly, the analyte(s) in the fluid move from the sample introduction area 201a on the proximal end to the PCR/detection area 201b on the distal end. Movement of the components and/or reaction mixture precursors from various reservoirs, e.g., first reservoir 205, second reservoir 207, third reservoir 211, and/or fourth reservoir 206 and into the fluidic channel 201 can be controlled in a similar manner such that the components and/or reaction mixture precursors can interact with the sample and/or analyte.

Elements that sense movement of the sample and/or analyte as the sample fluid flows through the fluidic channel can be added to the example apparatus 200 such as a piezoelectric or piezoresistive element to sense movement. These elements can be added to apparatus on one or both sides of the fluidic channel to sense up and down movement as well as left and right movement. Additionally, or alternatively, a sensing element could also be used to measure the elasticity (e.g., stiffness) of a sample like a cell to determine how hard or how soft it is. In some embodiments, the example apparatus 200 can include one or more chambers for mixing a sample with a reagent, e.g., a dried reagent and/or a liquid reagent.

The example apparatus 200 can further include one or more processors 215 coupled to, e.g., the one or more magnetic sensors 213, the temperature control device 209, and/or the first reservoir 205, second reservoir 207, third reservoir 211, and/or fourth reservoir. The one or more processors 215 can be coupled to other elements of the example apparatus 200 such as the opening 203, temperature controlled zones, etc. In at least one embodiment, the processor 215 can be an external control circuit connected to the example apparatus 200.

In some embodiments, pumping elements can be added to the example apparatus 200 in order to draw the sample and/or analyte to the magnetic sensors. Pumping can be achieved with a simple syringe operated manually, or with a micro-pump (e.g., a small micro-machined pump of dimension comparable to the analysis unit), a membrane pump, or with any other type of pumping system capable of producing sufficient suction to draw the sample and/or analyte in the example apparatus 200. Such pumping elements can be located at various positions along the fluidic channel 101, such as in the sample introduction area 201a and the PCR/detection area 201b. For example, pumping elements can be placed between the sample introduction area 201a and the PCR/detection area 201b to pump the sample from the opening 103 to the magnetic sensors. The example apparatus 200 can be powered by any means including but not limited to batteries, AC power supply, DC power supply, and the like.

As described above, an oligonucleotide probe can be chemically attached on or near a surface of the sensor. Additionally, or alternatively, the oligonucleotide probes can be adsorbed and/or electrostatically bound to the sensor. A variety of chemistries can be used for nucleotide surface hybridization. Non-limiting examples include, but are not limited to, amine-based chemistry, thiol-based chemistry, aldehyde-based chemistry, epoxy-based chemistry, carboxyl-based chemistry, amide-based chemistry, hydrazide-based chemistry, aldehyde-based chemistry, and hydroxy-based chemistry.

As an example, a thiol-modified oligonucleotide probe can be bound to gold (Au) or a silane (e.g., 3-glycidyloxypropyltriethoxysilane (GOPTS)). The gold can be deposited onto an oxide layer surface, such as a silicon oxide, by first depositing a Ti layer as an adhesive layer followed by deposition of a gold layer. With respect to the silane (e.g., GOPTS), the substrate can be rinsed with 95% ethanol for 5 min prior to being immersed in a solution of 2% GOPTS in 95% ethanol. The thiol-modified oligonucleotide probe can then be deposited using, e.g., laser induced forward transfer (LIFT), spotting, ink-jet printing, and/or photolithography. See, e.g., G. Tsekenis et al., “Surface functionalization studies and direct laser printing of oligonucleotides toward the fabrication of a micromembrane DNA capacitive biosensor,” Sensors and Actuators B: Chemical, vol. 175, pp. 123-131, December 2012.

As another example of chemically attaching an oligonucleotide probe on or near a surface of the sensor, oxide layer surface (such as a silicon oxide surface) can be treated with an aminosilane resulting in a layer of primary amines and/or epoxides. Oligonucleotides modified with an NH₂ group can be immobilized onto epoxy silane-derivatized or isothiocyanate coated silicon surfaces. Succinylated oligonucleotides can be coupled to aminophenyl- or aminopropyl-derivitized silicon surfaces by peptide bonds, and disulfide-modified oligonucleotides can be immobilized onto a mercapto-silanized silicon surfaces by a thiol/disulfide exchange reactions or through chemical cross linkers. Various modifiers can be used on the oligonucleotide probe such as 5′ amino modifiers (which are added to the 5′-terminus of a target oligonucleotide), 3′-amino modifiers, internal amino modifiers, 3′-thiol modifiers, 5′-thiol modifiers, acrydite modifiers. Having such modifiers allows attachment of the oligonucleotide to various oxide (such as silicon oxide) surfaces. 5′-amino modifiers can include β-cyanoethyl phosphoramidites, simple amino groups with a six or twelve carbon spacers, amino modified thymidine, and cytosine. 5′-thiol modifiers can include S-trityl-6-mercaptohexyl derivatives. Acrydite modifiers can include acrylic acid modifying groups attached to the 5′ end of the oligonucleotide. Procedures for modification of surfaces with the modified oligonucleotide probes are known in the art. See, e.g., “Strategies for Attaching Oligonucleotides to Solid Supports,” Integrated DNA Technologies, 2014 (v₆), pp. 1-22.

### Example Operations

As described above, and in some embodiments, the apparatus described herein (e.g., example apparatus 100 and example apparatus 200) can perform one or more of the following operations: for RNA virus detection: pretreatment to lyse the cell; RNA extraction to remove RNA from the cellular material; reverse transcription of the RNA to a complementary DNA; PCR to amplify the DNA; and detection of the DNA. For DNA virus detection, and in some embodiments, reverse transcription can be “turned off” in apparatus and RNA extraction can be replaced by DNA extraction from the cellular material. Other operations can include, but are not limited to, diluting and/or concentrating the sample and/or analyte with a liquid, mixing the sample and/or analyte with a material, and a combination thereof. Non-limiting examples of each of these operations is provided below with respect to the example apparatus 100 of FIG. 1A.

Similar operations and components can be used with the example apparatus 150 of FIG. 1B and example apparatus 200 of FIG. 2.

After the sample is placed at the opening (e.g., opening 103) of the fluidic channel 101, the sample can undergo a pretreatment operation. Pretreatment of the sample can include introducing pretreatment components and/or reaction mixture precursors that, upon interaction with the sample comprising cells and cellular material, lyse the cell.

These pretreatment components and/or reaction mixture precursors can be released from reservoir 105a and into to the sample introduction area 101a of the fluidic channel 101 in order to interact with the sample. Cell lysis can be achieved by physical and/or chemical methods. Chemical methods to lyse the cell can include the use of various reagents such as lytic enzymes (such as Proteinase K), chaotropic agents, and different types of detergents. Physical methods of lysing the cell can include grinding, shearing, bead beating, and shocking. Shock waves created by rapid changes in pressure elicited by sonication or cavitation can be used. Shearing can be performed by applying a tangential force to make a hole in the cell. Bead beating can be performed by using, e.g., glass and/or steel beads to rupture the cell wall. Depending on the type of sample, e.g., serum, blood, plasma, sputum, etc., chemical and/or physical methods can be used. Osmotic shock can also be used. Combinations of different reagents can be used as well. For example, enzymatic lysis can be performed by using proteases to free the nucleic acid. The proteases can, optionally, be used with one or more detergents, one or more buffers, and one or more salts (e.g., chaotropic salts) to facilitate solubilization of the cell wall and/or cell membrane. Heat can be added to aid in lysing the cell.

As an example, an enzyme (with or without buffer, salts, and/or detergents) can be mixed with the sample in the fluidic channel and incubated at a temperature of about 50-70° C. Higher temperatures such as from about 75-95° C. can be used for blood samples. Sputum samples can optionally be mixed with dithiothreitol at temperatures of about 30-45° C. prior to mixing the sample with the enzyme, detergent, salt, and/or buffer. Dithiothreitol can be used to release cells from mucus.

Following cell lysis, the nucleic acid extraction operation can include introducing extraction components and/or reaction mixture precursors that, upon interaction with the sample, extract the analyte of interest from the cellular material. These extraction components and/or reaction mixture precursors can be released from reservoir 105b into the fluidic channel 101 in order to interact with the sample.

Non-limiting examples of extraction components and/or reaction mixture precursors can include solid-phase extraction agents, such as silica matrices, glass particles, diatomaceous earth, magnetic beads, anion exchange resins, cellulose matrices, and combinations thereof. Elution from these materials can be performed using buffers. Silicates can be used along with salt and alkaline conditions (e.g., alkaline Tris-EDTA) to bind nucleic acids. The silicate can be in the form of a gel or glass particle such as a glass microfiber. Glass particles can be in a powder and/or microbead form. For anion exchange materials, a resin such as diethylaminoethyl cellulose can be used to attract the negatively charged phosphate of the nucleic acid.

Appropriately selected buffers (e.g., selected by pH and/or salt concentration) can be used to aid in binding the nucleic acid to the solid-phase extraction agents and for eluting the nucleic acid from the solid-phase extraction agents. Detergents, and/or chelating agents, such as the Tris pH 8, sodium dodecyl sulfate (SDS), and/or ethylenediaminetetraacetic acid (EDTA), can optionally be used with the buffers to aid in binding the nucleic acid to the solid-phase extraction agents and for eluting the nucleic acid from the solid-phase extraction agents.

As a non-limiting example, magnetic particles, such as magnetic glass particles (MGP), that bind specifically to nucleic acids can be used to extract the nucleic acid from the cellular material. The magnetic particles are functionalized such that they selectively interact with and/or bond to a desired nucleic acid. Such magnetic glass particles immobilize the nucleic acids on the MGP surfaces. The MGPs can be used along with a buffer. The MGPs, having bound the nucleic acid can aggregate via magnetization (using, e.g., a small neodymium magnet and/or solenoid) along the fluidic channel prior to reaching temperature control device 109 or the area where PCR reagents are released from second reservoir 207. The extracted nucleic acid can then be eluted from the MGPs using a suitable eluent such as those buffers listed above to elute the nucleic acid from the solid-phase extraction agent. Detergents and/or chelating agents can also be used during the extraction operation.

For RNA viruses, reverse transcription can then be performed on the extracted nucleotide. Reverse transcription is a procedure catalyzed by an enzyme, reverse transcriptase, that synthesizes a complementary DNA (cDNA) from a single stranded RNA molecule, with the use of, e.g., oligonucleotide primers having free 3′-hydroxyl groups.

Performing a reverse transcription operation on the nucleotide can include introducing reverse transcription components and/or reaction mixture precursors that, upon interaction with the sample, perform a reverse transcription reaction on the RNA. These reverse transcription components and/or reaction mixture precursors can be released from fourth reservoir 106. Non-limiting examples of reverse transcription components and/or reaction mixture precursors, can include reverse transcriptase, primers (such as oligo(dT) primers, random primers, and/or gene-specific primers), buffers, and/or inhibitors. Salt concentrations and pH can be adjusted depending on the application to maintain favorable ionic strength and pH for the reverse transcription reaction. The temperature can also be adjusted to modulate the creation of cDNA from RNA.

Following reverse transcription, the nucleotide (e.g., cDNA) to be amplified travels toward PCR area 101b. At PCR area 101b, the nucleotide undergoes a PCR operation. PCR relates to a procedure whereby a limited segment of a nucleic acid molecule, which frequently is a desired or targeted segment, is amplified repetitively to produce a large amount of DNA molecules of that segment. The procedure can depend on repetition of a large number of priming and transcription cycles. In each cycle, two oligonucleotide primers bind to the segment, and define the limits of the segment. A primer-dependent DNA polymerase then transcribes, or replicates, the strands to which the primers have bound. Thus, in each cycle, the number of DNA duplexes is doubled.

Performing a PCR operation on the sample, e.g., the nucleotide, can include introducing PCR components and/or reaction mixture precursors that, upon interaction with the sample, amplify DNA. The PCR components and/or reaction mixture precursors can be released from second reservoir 107 and into PCR area 101b in order to interact with the sample. The PCR components and/or reaction mixture precursors can include PCR reagents that promote amplification of a target nucleic acid sequence (e.g., cDNA). Such PCR reagents can include primers (e.g., forward primers and reverse primers), enzymes such as polymerase (e.g., DNA polymerase) or polymerases with exonuclease activity, substrates such as nucleic acids and oligonucleotide primers, and/or buffers. The primer is a pair of short nucleotides sequences (e.g., 10-20 base pairs) corresponding to the partial sequences at the two extremities of the specific amplified DNA sequence. DNA polymerase is an enzyme that enables creation of the new DNA strand from single-stranded DNA as a template by assembling specifically the nucleotides.

The polymerase chain reaction generally comprises temperature cycles between, e.g., two or three temperatures (or more), for each PCR phase—denaturation phase, annealing phase, and extension phase. The temperature cycles can be controlled by the temperature control device 109. The denaturation phase can be performed at a temperature above about 90° C., such as about 94° C. to about 98° C. Denaturation causes DNA melting, e.g., the separation of the DNA strands yielding two separate single-stranded DNA. The annealing phase can be performed at a temperature of about 50° C. to about 65° C. to allow annealing of the primers (e.g., the forward and/or reverse primers) to the single-stranded DNA templates at specific locations corresponding to the two extremities of the specific amplified DNA sequence. The annealing phase can be made at the same temperature as the extension phase to make a PCR using only two different temperatures. The extension phase can be performed at a temperature of about 65° C. to about 75° C., such as about 70° C. or about 72° C. The temperature can depend on the DNA polymerase used since it corresponds to the phase where the DNA polymerase binds to the primer/single-strained DNA complex to synthesize the new complementary DNA strand by incorporating the surrounding nucleotides. In some embodiments, the annealing phase can be performed at the same temperature as the extension phase to, and such PCR operations can use only two different temperatures.

As another example of the polymerase chain reaction, the DNA double helix can be melted at a temperature greater than about 90° C. and the DNA strands separate. The temperature can then be decreased to slightly below the melting temperature of the of the primers used (e.g., the forward and/or reverse primers). The primers bind to the available strands. The forward and/or reverse primers can be provided to the fluidic channel in excess to ensure that the strands do not come back and reanneal to one another. Polymerization (or extension) can occur using DNA polymerase in the 5′ to 3′ direction on each strand. The incorporated additional nucleotides give rise to new strands that extend past the sequence of interest. The polymerized strands can then act as a template for the other primer (if forward primer bound first, reverse primer now binds, and vice-versa). Polymerization occurs again, this time ending at the sequence of interest. The incorporated additional nucleotides give rise to new strands that only encode the sequence of interest. The synthesized strands encoding the sequence of interest then anneal to one another to form the end product to be detected by the one or more magnetic sensors 113. A result of the PCR is to amplify the nucleotide sequence exponentially up to detectable quantities.

### Magnetic Detection

Following amplification of the nucleotide, functionalized magnetic nanoparticles (MNPs) can bind to the nucleotide to form a MNP-tagged nucleotide sequence. Forming the MNP-tagged nucleotide can include introducing MNP components and/or reaction mixture precursors that, upon interaction with the sample, react a nucleotide sequence with a functionalized MNP. MNP components and/or reaction mixture precursors can be released from third reservoir 111. The MNP-tagged nucleotide can then be detected by one or more of magnetic sensors 113. That is, the one or more magnetic sensors 113 detect the presence or absence of a magnetic particle.

In some embodiments, the detection can be performed by thermal detection of the magnetic nanoparticles. Accordingly, magnetic can refer to the sensor being magnetic and/or the sensor detecting the effects of magnetism, which are not strictly limited to magnetic field detection.

Various magnetic detection devices, methods, and/or systems can be employed, e.g., U.S. patent application Ser. No. 16/659,383, filed Oct. 21, 2019 and entitled “MAGNETORESISTIVE SENSOR ARRAY FOR MOLECULE DETECTION AND RELATED DETECTION SCHEMES”; U.S. patent application Ser. No. 16/697,013, filed Nov. 26, 2019 and entitled “THERMAL SENSOR ARRAY FOR MOLECULE DETECTION AND RELATED DETECTION SCHEMES”; U.S. patent application Ser. No. 16/727,064, filed Dec. 26, 2019 and entitled “DEVICES AND METHODS FOR MOLECULE DETECTION BASED ON THERMAL STABILITIES OF MAGNETIC NANOPARTICLES”; U.S. patent application Ser. No. 16/791,759, filed Feb. 14, 2020 and entitled “SPIN TORQUE OSCILLATOR (STO) SENSORS USED IN NUCLEIC ACID SEQUENCING ARRAYS AND DETECTION SCHEMES FOR NUCLEIC ACID SEQUENCING”; U.S. patent application Ser. No. 16/819,636, filed Mar. 16, 2020 and entitled “DEVICES AND METHODS FOR FREQUENCY- AND PHASE-BASED DETECTION OF MAGNETICALLY-LABELED MOLECULES USING SPIN TORQUE OSCILLATOR (STO) SENSORS;” and U.S. patent application Ser. No. 16/823,592 filed Mar. 19, 2020 and entitled “MAGNETIC GRADIENT CONCENTRATOR/RELUCTANCE DETECTOR FOR MOLECULE DETECTION,” each of which is incorporated herein by reference in their entireties.

### A. MNPs

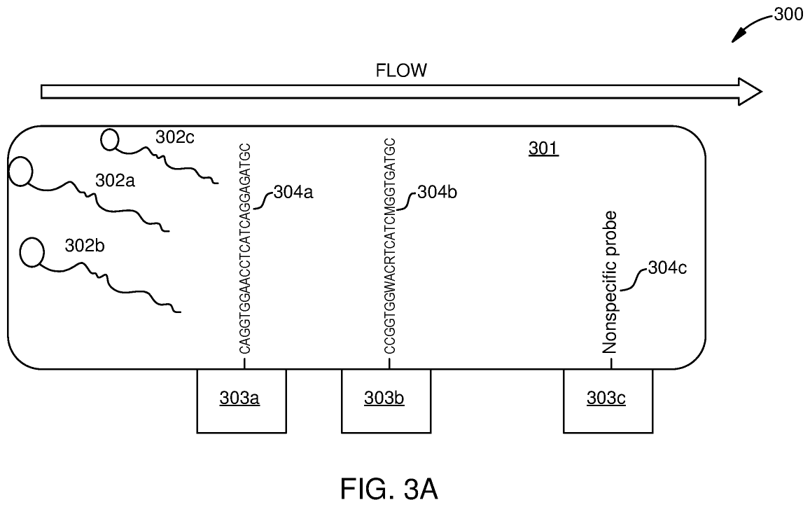

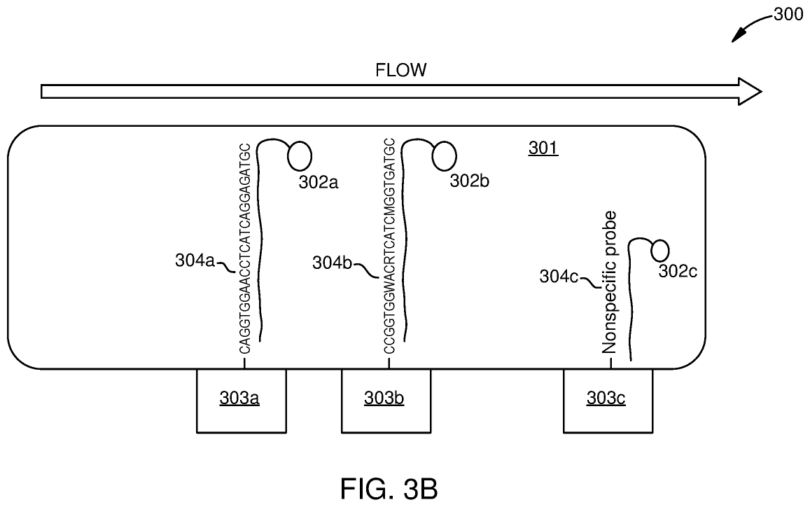

In some embodiments, MNPs that are suitable for use herein can have a wide range of sizes (e.g., tens to hundreds of nanometers (nm)) and shapes (e.g., spherical, cubic, pyramidal, etc.). The magnetism in these particles is due to exchange interactions in the materials that align unpaired core electrons in the material's lattice in the same direction, resulting in a net moment of angular momentum in the material that is also called the magnetic moment or magnetization of the nanoparticle. (The terms “magnetic moment” and “magnetization” are used interchangeably herein.) The magnetic moment (a dipole moment within an atom that originates from the angular momentum and spin of electrons) of the MNP, at least at some temperatures, gives rise to magnetic fields that, as described further below, can be used to detect the presence of the MNP. Additional magnetic anisotropy energies (e.g., magnetocrystalline, demagnetization) also help define stable orientations for the magnetization of the MNP, so that, when the spatial orientation of a particle is well defined, so is the magnetization direction of the particle. FIG. 3 illustrates an exemplary MNP having a magnetization that is illustratively fixed along the z-axis due to magnetic anisotropy. It is to be understood that if the particle is mechanically rotated, so is the magnetization direction.

In some embodiments, the magnetization of a MNP can be temperature-dependent, and for MNPs that are sufficiently small, there can be two temperatures at which their magnetic properties change significantly. The first change occurs at what is referred to as the blocking temperature, denoted as TB. When a nanoparticle is sufficiently small, its magnetization can flip direction randomly in the absence of an external magnetic field. At this point, the thermal energy of the particle (kT) is sufficiently larger than the anisotropy energies such that the magnetization no longer points along a single axis. Instead, the magnetization can randomly flip or rotate between two or more orientations, at which point the MNP transitions from being ferromagnetic to being considered superparamagnetic. Magnetic nanoparticles are said to be “superparamagnetic” when the loop area of their hysteresis loop, when measured under quasi-static conditions, is zero, which occurs when the nanoparticle cores are small enough to support only one magnetic domain per core, in which case they are single-domain particles.

The magnetic properties of a MNP can also change at or around the Curie temperature, denoted as Tc, which is greater than or equal to the blocking temperature TB. The Curie temperature (sometimes referred to as the ferromagnetic transition temperature) is the temperature above which ferromagnetic materials lose their permanent magnetic properties and become paramagnetic. Ferromagnetic and paramagnetic materials have different intrinsic magnetic moment structures, and these properties change at a material's Curie temperature. Stated another way, ferromagnetism appears only below the Curie temperature. At temperatures above the Curie temperature, the thermal energies are large enough to overcome the exchange interactions amongst the core electrons and eliminate the net moment of the MNP. Consequently, the ordered magnetic moments change and become disordered (e.g., oriented randomly, resulting in a zero net magnetization). Below the Curie temperature, a MNP is ferromagnetic (has moments that are magnetized spontaneously) and, as such, generates a magnetic field, but above the Curie temperature, the particle is in a paramagnetic state and does not generate a spontaneous magnetic field of its own.

The inventor of the present disclosure had the insight that the differences in MNPs' magnetic properties around the blocking temperature and around the Curie temperature can be exploited for molecule detection, as explained in further detail below.

As will be appreciated by those having ordinary skill in the art, there are many suitable MNPs that can be used with the systems and methods described below. For example, the Curie temperature of a MNP can be adjusted over a small range between room temperature and 100° C. by changing the composition of compounds. For example, Y₃Fe₅₋ₓAlₓO₁₂ particles with an average diameter of 100 nm and with Curie temperatures varying from 7 to 140° C. by varying the aluminum content in the MNP have been synthesized. See, e.g., Grasset et al., “Synthesis, magnetic properties, surface modification and cytotoxicity evaluation of Y₃Fe₅₋ₓAlₓO₁₂ garnet submicron particles for biomedical applications,” JMMM, 234 (2001) 409-418. La₁₋ₓSrₓMn_{1-y}Ti_{y}O₃ particles having Curie temperatures between 20 and 90° C. by varying the titanium content have also been synthesized. See, e.g., Phuc et al., “Tuning of the Curie Temperature in La₁₋ₓSrₓMn_{1-y}Ti_{y}O₃,” Journal of the Korean Physical Society, Vol. 52, No. 5, May 2008, pp. 1492-1495. Finally, GdSi alloys doped with elements such as germanium, erbium, and rhodium have shown Curie temperatures in a range around room temperature with smaller (approximately 40 nm diameter) sizes and more regular spherical shapes. See, e.g., Alnasir et al., “Magnetic and magnetothermal studies of pure and doped gadolinium silicide nanoparticles for self-controlled hyperthermia applications,” JMMM, 449 (2018) 137-144. These examples represent only a small sample of possible materials for generating MNPs suitable for use with the disclosed embodiments and are not meant to be limiting. Those having skill in the art will understand that many MNPs having the properties described in this disclosure exist or can be developed without substantial experimentation. Moreover, those skilled in the art will recognize that the blocking temperature can also be adjusted by doping, for example, by changing the crystalline structure of a material.

Once the MNPs have been selected, there are a number of ways to attach the MNPs to the molecules to be detected and (if applicable) to cleave the MNPs following detection. For example, the MNPs can be attached to a base or a molecule to be detected, in which case the MNPs can be cleaved chemically. As another example, the MNPs can be attached to a phosphate, in which case the MNPs can be cleaved by, for example, polymerase or, if attached via a linker, by cleaving the linker.

In some embodiments, the MNP can be linked to the nitrogenous base (e.g., A, C, T, G, or a derivative) of the nucleotide precursor. After incorporation of the nucleotide precursor and detection by a detection device (e.g., as described below), the MNP can be cleaved from the incorporated nucleotide.

In some embodiments, the MNP can be attached to the nucleotide via a cleavable linker. Cleavable linkers are known in the art and have been described, e.g., in U.S. Pat. Nos. 7,057,026, 7,414,116 (each of which is hereby incorporated by reference in its entirety for all purposes) and continuations and improvements thereof. In some embodiments, the MNP can be attached to the 5-position in pyrimidines or the 7-position in purines via a linker comprising an allyl or azido group. In some embodiments, the linker can include a disulfide, indole, a Sieber group, a t-butyl Sieber group, and/or a dialkoxybenzyl group. The linker can further contain one or more substituents selected from alkyl (such as C₁₋₆) groups, alkoxy (such as C₁₋₆) groups, nitro groups, cyano groups, fluoro groups, and/or groups with similar properties. Briefly, the linker can be cleaved by water-soluble phosphines and/or phosphine-based transition metal-containing catalysts. Other linkers and linker cleavage mechanisms are known in the art. For example, linkers comprising trityl groups, p-alkoxybenzyl ester groups, p-alkoxybenzyl amide groups, tert-butyloxycarbonyl (Boc) groups, and acetal-based groups can be cleaved under acidic conditions by a proton-releasing cleavage agent such as an acid. A thioacetal or other sulfur-containing linker can be cleaved using a thiophilic metals, such as nickel, silver, and/or mercury. The cleavage protecting groups can also be considered for the preparation of suitable linker molecules. Ester- and disulfide-containing linkers can be cleaved under reductive conditions. Linkers containing triisopropyl silane (TIPS) or t-butyldimethyl silane (TBDMS) can be cleaved in the presence of fluoride (F) ions. Photocleavable linkers cleaved by a wavelength that does not affect other components of the reaction mixture can include linkers comprising o-nitrobenzyl groups. Linkers comprising benzyloxycarbonyl groups can be cleaved by Pd-based catalysts.

In some embodiments, the nucleotide comprises a MNP label attached to a polyphosphate moiety as described in, e.g., U.S. Pat. Nos. 7,405,281 and 8,058,031, each of which is hereby incorporated by reference in its entirety for all purposes. Briefly, the nucleotide can comprises a nucleoside moiety and a chain of 3 or more phosphate groups where one or more of the oxygen atoms are optionally substituted, e.g., with S. The label can be attached to the α, β, γ or higher phosphate group (if present) directly or via a linker. In some embodiments, the MNP label can be attached to a phosphate group via a non-covalent linker as described, e.g., in U.S. Pat. No. 8,252,910, which is hereby incorporated by reference in its entirety for all purposes. In some embodiments, the linker is a hydrocarbon selected from substituted or unsubstituted alkyl, substituted or unsubstituted heteroalkyl, substituted or unsubstituted aryl, substituted or unsubstituted heteroaryl, substituted or unsubstituted cycloalkyl, and substituted or unsubstituted heterocycloalkyl, or a combination thereof; see, e.g., U.S. Pat. No. 8,367,813, which is hereby incorporated by reference in its entirety for all purposes. The linker can also comprise a nucleic acid strand; see, e.g., U.S. Pat. No. 9,464,107, which is hereby incorporated by reference in its entirety for all purposes.

In embodiments in which the MNP is linked to a phosphate group, the nucleotide can be incorporated into the nascent chain by the nucleic acid polymerase, which also cleaves and releases the detectable MNP. In some embodiments, the MNP can be removed by cleaving the linker, e.g., as described in U.S. Pat. No. 9,587,275, which is hereby incorporated by reference in its entirety for all purposes.

After attaching the MNP to the nucleotide sequence to form the MNP-tagged nucleotide sequence, the nucleotide sequence can be detected by magnetic detection as described below.

### B. Detection Methods

Various magnetic detection methods and systems can be used for embodiments described herein. See, e.g., U.S. patent application Ser. No. 16/659,383, filed Oct. 21, 2019; U.S. patent application Ser. No. 16/697,013, filed Nov. 26, 2019; U.S. patent application Ser. No. 16/727,064, filed Dec. 26, 2019; U.S. patent application Ser. No. 16/791,759, filed Feb. 14, 2020; U.S. patent application Ser. No. 16/819,636, filed Mar. 16, 2020; and U.S. patent application Ser. No. 16/823,592 filed Mar. 19, 2020, each of which is incorporated herein by reference in their entireties.

### Example Detection Sequence

FIGS. 3A and 3B show an example detection area 300 according to at least one embodiment of the present disclosure. The example detection area 300 can correspond to the detection area 101c of example apparatus 100. Generally, as the MNP-tagged nucleotide flow into the detection area, the oligonucleotide probe attached to magnetic sensor will interact with the MNP-tagged nucleotide. As the MNP-tagged nucleotide interacts with probe, the MNP is brought close to one of the magnetic sensors and a determination of the nucleotide present can be made.

Referring to FIG. 3A, the MNPs are bound to the nucleotide (DNA) fragments as MNP-tagged nucleotides 302a, 302b, 302c. For this example, MNP-tagged nucleotide 302a can be indicative of Wuhan-CoV, MNP-tagged nucleotide 302b can be indicative of SARS-CoV, bat-SARS-related COVs, and Wuhan-CoV, and MNP-tagged nucleotide 302c is the MNP-tagged non-specific nucleotide. The example detection area 300 can include one or more magnetic sensors 303 along the fluidic channel 301, such as magnetic sensor 303a, magnetic sensor 303b, and magnetic sensor 303c. The magnetic sensors 303 can be configured to detect the presence or absence of a magnetic nanoparticle. Each magnetic sensor 303 can, independently, have a oligonucleotide probe 304 chemically attached to the magnetic sensor 303, and each oligonucleotide probe 304 can be, independently, selective for a specific MNP-tagged nucleotide (e.g., MNP-tagged nucleotide 302a or MNP-tagged nucleotide 302b) or the non-specific MNP-tagged nucleotide (e.g., MNP-tagged nucleotide 302c).

For example, magnetic sensor 303a can include an oligonucleotide probe 304a specific for Wuhan-CoV and will not detect SARS-CoV, while magnetic sensor 303b can include oligonucleotide probe 304b specific for SARS-CoV, bat-SARS-related COVs, and Wuhan-CoV. For oligonucleotide probe 304b, W=A/T, R=G/A, and M=A/C. The example detection area 300 can also include at least one magnetic sensor 303c that can include an oligonucleotide probe 304c that is not specific for any particular MNP-tagged nucleotide. Magnetic sensor 303c can serve to signal that the process has been completed and can ensure that DNA was present for the test.

FIG. 3B shows the example detection area 300 after the MNP-tagged nucleotides 302a, 302b, and 302c are attached (chemically or physically) to the oligonucleotide probes 304a, 304b, and 304c, respectively. As the MNP-tagged nucleotide 302 interacts with the respective oligonucleotide probe 304, the MNP of the MNP-tagged nucleotide 302 is brought close to the respective magnetic sensor 303 and a determination of the nucleotide present can be made.

### Example Real-Time Detection Sequence

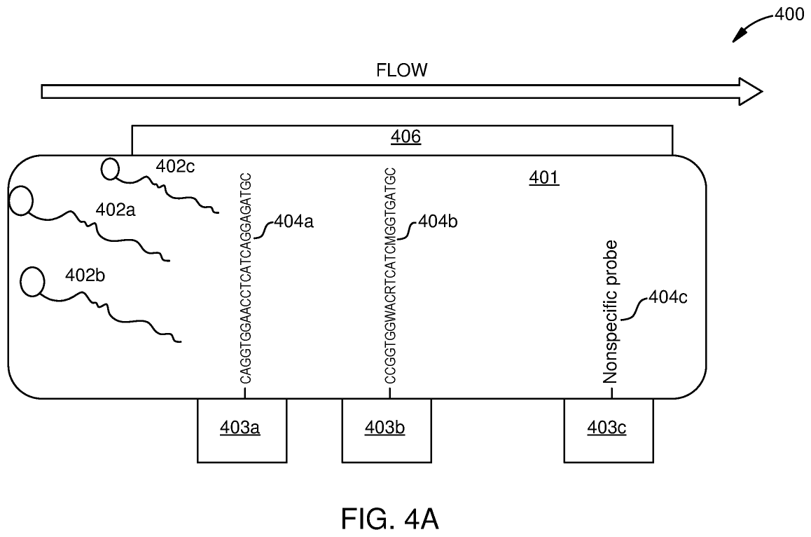

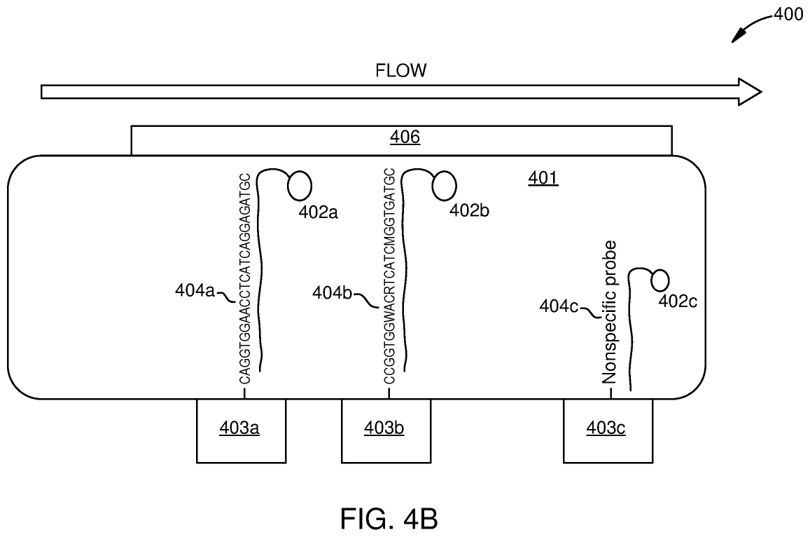

FIGS. 4A and 4B show an example detection area 400 according to at least one embodiment of the present disclosure. FIGS. 4A and 4B illustrate real time detection during the PCR thermal cycling process. The example detection area 400 can correspond to the PCR/detection area 201b of example apparatus 200. Generally, as the MNP-tagged nucleotide flow into the detection area, the oligonucleotide probe attached to magnetic sensor will interact with the MNP-tagged nucleotide. As the MNP-tagged nucleotide interacts with probe, the MNP is brought close to one of the magnetic sensors and a determination of the nucleotide present can be made.

Referring to FIG. 4A, the MNPs are bound to the nucleotide (DNA) fragments as MNP-tagged nucleotides 402a, 402b, 402c. For this example, MNP-tagged nucleotide 402a can be indicative of Wuhan-CoV, MNP-tagged nucleotide 402b can be indicative of SARS-CoV, bat-SARS-related COVs, and Wuhan-CoV, and MNP-tagged nucleotide 402c is the MNP-tagged non-specific nucleotide. The example detection area 400 can include one or more magnetic sensors 403 along the fluidic channel 401, such as magnetic sensor 403a, magnetic sensor 403b, and magnetic sensor 403c. The magnetic sensors 403 can be configured to detect the presence or absence of a magnetic nanoparticle. Each magnetic sensor 403 can, independently, have a oligonucleotide probe 404 chemically attached to the magnetic sensor 403, and each oligonucleotide probe 404 can be, independently, selective for a specific MNP-tagged nucleotide (e.g., MNP-tagged nucleotide 402a or MNP-tagged nucleotide 402b) or the non-specific MNP-tagged nucleotide (e.g., MNP-tagged nucleotide 402c).

For example, magnetic sensor 403a can include an oligonucleotide probe 404a specific for Wuhan-CoV and will not detect SARS-CoV, while magnetic sensor 403b can include oligonucleotide probe 404b specific for SARS-CoV, bat-SARS-related COVs, and Wuhan-CoV. For oligonucleotide probe 404b, W=A/T, R=G/A, and M=A/C. The example detection area 400 can also include at least one magnetic sensor 403c that can include an oligonucleotide probe 404c that is not specific for any particular MNP-tagged nucleotide. Magnetic sensor 403c can serve to signal that the process has been completed and can ensure that DNA was present for the test.

At higher temperatures during the denaturation phase of PCR, the DNA can denature. The temperature can be controlled by temperature control device 406. As shown in FIG. 4A, the MNP-tagged nucleotides 402 are no longer attached to the respective oligonucleotide probes 404 at the higher temperatures. Although not shown, the MNP may also not be attached to the nucleotide. As shown in FIG. 4B, the temperature can be lower during the annealing and elongation phases (as controlled by temperature control device 406). Here, the nucleotides will anneal and elongate, reattach to the MNP, and form the respective MNP-tagged nucleotide 402. The MNP-tagged nucleotide at the lower temperature can attach (chemically and/or physically) to the respective oligonucleotide probe 404. As the MNP-tagged nucleotide 402 interacts with the respective oligonucleotide probe 404, the MNP of the MNP-tagged nucleotide 402 is brought close to the respective magnetic sensor 403 and a determination of the nucleotide present can be made. At each PCR cycle, a measurement can be taken and the presence of the MNP-tagged nucleotide at the sensor can be monitored real-time.

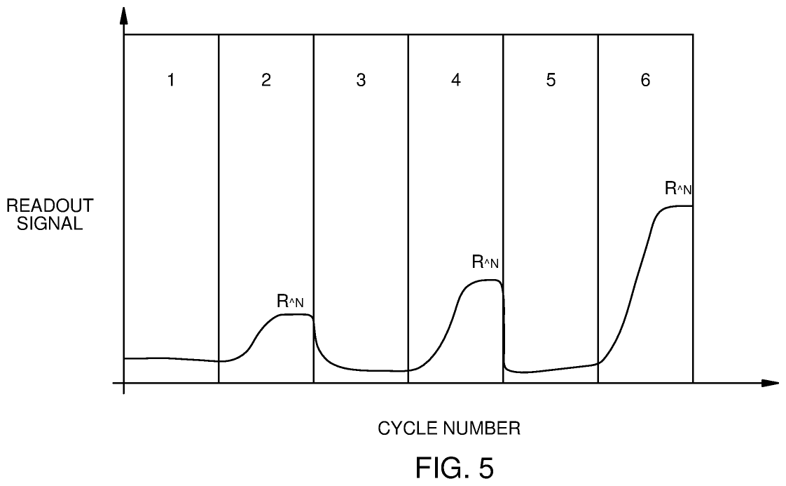

FIG. 5 shows an example readout signal 500 during PCR according to at least one embodiment of the present disclosure. One or more processors, e.g., one or more processors 115 or one or more processors 215, can receive a signal from the one or more magnetic sensors and process such signals to provide results and/or to control the process to take actions. R_{N} is the amount of nucleotide detected and therefore can provide a quantitative measurement of the nucleotide. Areas 1, 3, and 5 of the correspond to denaturation phases of the PCR, and areas 2, 4, and 6 correspond to annealing and elongation phases. At area 1, the first denaturation phase, the existing nucleotide is denatured and is amplified by the polymerase. Upon cooling down at area 2, the first annealing and elongation phases, the nucleotide can attach to the MNP and to the sensor via the probe. Attachment to the MNP brings the nucleotide closer to the sensor and leads to an increase in the readout signal R_{N}. At area 3, the second denaturation phase, the nucleotide can detach from the MNP and the sensor, leading to a decrease in readout signal R_{N}. The nucleotide is also amplified at area 3. Upon cooling down at area 4, the second annealing and elongation phases, the nucleotide can attach to the MNP and to the sensor via the probe. Attachment to the MNP brings the nucleotide closer to the sensor and leads to an increase in the readout signal R_{N}. These cycles, 1-2, 3-4, and so forth, continue for as many cycles as desired. A baseline correction can be performed an the readout signal.

In some embodiments, a baseline correction can be performed. Baseline correction can permit removal of variations in sensitivity, temperature, chemicals, electronics, environmental effects (external magnetic field), contamination (e.g., due to the MNPs), etc. The baseline correction can utilize a detector with a oligonucleotide probe specific for a certain sequence, a detector without an oligonucleotide probe, and/or a detector with a non-specific oligonucleotide probe. In at least one embodiment, a ratio of the target sequence detected by the specific probe versus the sequence detected by the non-specific oligonucleotide probe can be determined. Such a ratio can effectively remove, e.g., baseline magnetic detection.

For example, if there are some MNPs in the fluid that are not attached to a nucleotide, the MNPs can affect all sensors equally. The target MNP-tagged nucleotide, on the other hand, only affects the target sensor. By relating the signal measured at the target sensor to other sensors, such effects can be excluded. According to certain embodiments, the at least one processor (e.g., one or more processors 115 and/or one or more processors 215) can be configured to perform a baseline correction. The baseline correction can include determining a baseline signal based on the signal(s) detected and removing at least a portion of the baseline signal from the signal(s) detected to generate an adjusted signal. In some embodiments, the baseline correction can be performed by the processor receiving a plurality of signals and removing at least a portion of the plurality of signals to generate an adjusted signal.

Additionally, or alternatively, an absolute concentration of the target nucleotide can be determined. In such embodiments, an internal standard can be employed. The internal standard can be a different nucleotide (nucleotide_{REF}) that undergoes amplification and detection operations as the target oligonucleotide. The nucleotide_{REF} can be detected by a detected with a reference probe, and the absolute concentration of the target nucleotide can be estimated with respect to the signal strength produced from the reference detector.

### Methods of Using the Magnetic PCR Assay

The present disclosure is also generally related to methods of using the magnetic PCR assay. For example, the magnetic PCR assay can be used to determine the presence of a component of interest within a sample, e.g., a nucleic acid, a virus, a bacterium, a protein, an antibody, etc. These components can be minor constituents of the sample and can therefore be challenging to detect by conventional methods. Moreover, any molecule type that can be labeled by a MNP can be detected using the methods and detection devices disclosed herein. Thus, the detection methods can extend to inorganic molecules or non-living molecules, such as contaminants, minerals, chemical compounds, etc.

In one example, a method for detecting an analyte (e.g., a nucleotide) can include introducing a sample to magnetic PCR assay described herein. Introducing can include inserting a swab to an opening of the assay, pipetting a sample into an opening of the assay (e.g., an opening of the fluidic channel). The method can further include performing processes to the sample (or compound, or analyte) in a fluidic channel of the apparatus described herein, and detecting the analyte.

According to at least one embodiment, one or more operations of the apparatus (e.g., assay) and methods described above can be included as instructions in a computer-readable medium for execution by a control unit (e.g., one or more processors 115 and/or processor 215) or any other processing system. The one or more operations that can be performed can include, but are not limited to, pretreatment to lyse the cell; RNA extraction to remove RNA from the cellular material; reverse transcription of the RNA to a complementary DNA; PCR to amplify the DNA; detection of the DNA, or a combination thereof. Other operations that can be performed can include, but are not limited to, diluting the sample and/or analyte with a liquid, concentrating the sample, separating the sample from debris, mixing the sample and/or analyte with a material, and a combination thereof.

The computer-readable medium can include any suitable memory for storing instructions, such as read-only memory (ROM), random access memory (RAM), flash memory, an electrically erasable programmable ROM (EEPROM), a hard disk drive, a compact disc ROM (CD-ROM), a floppy disk, punched cards, magnetic tape, and the like.

### EMBODIMENTS LISTING

The present disclosure provides, among others, the following embodiments, each of which may be considered as optionally including any alternate embodiments.

Clause 1. An apparatus for detecting a nucleotide sequence, comprising:

a fluidic channel coupled to one or more reservoirs, the fluidic channel comprising a sample introduction component, a polymerase chain reaction component, and a detection component;

a temperature control device to heat or cool the fluidic channel;

one or more sensors to detect the presence or absence of a magnetic nanoparticle; and

a processor coupled to the one or more sensors and to the temperature control device.

Clause 2. The apparatus of Clause 1, wherein:

a first reservoir of the one or more reservoirs includes components to extract a nucleotide from cellular material;

a second reservoir of the one or more reservoirs includes PCR reagents to amplify a first nucleotide sequence; and

a third reservoir of the one or more reservoirs includes components to react a second nucleotide sequence with a magnetic nanoparticle (MNP).

Clause 3. The apparatus of any one of Clause 1 or Clause 2, wherein a fourth reservoir of the one or more reservoirs includes components to perform reverse transcription.

Clause 4. The apparatus of any one of Clauses 1-3, wherein at least one sensor of the one or more sensors includes a chemically-bound oligonucleotide probe.

Clause 5. The apparatus of any one of Clauses 1-4, wherein:

a first sensor of the one or more sensors configured to detect a first virus sequence;

a second sensor of the one or more sensors configured to detect a second virus sequence; and

a third sensor of the one or more sensors configured to detect a non-specific virus sequence.

Clause 6. The apparatus of any one of Clauses 1-5, further comprising:

one or more temperature-controlled zones;

one or more chambers for mixing a sample with a reagent;

an element for sensing the presence of a reagent, a fluid, or a combination thereof;

an element for controlling fluid motion; or a combination thereof.

Clause 7. The apparatus of any one of Clauses 1-6, wherein the element for controlling fluid motion comprises a piezoelectric pump, a coil, a membrane, electrodes, or a combination thereof.

Clause 8. The apparatus of any one of Clauses 1-7, wherein the processor is configured to perform a baseline correction by receiving a plurality of signals and removing at least a portion of the plurality of signals to generate an adjusted signal.

Clause 8. An apparatus for detecting a nucleotide sequence, comprising:

a fluidic channel coupled to one or more reservoirs, the fluidic channel comprising a sample introduction component, a polymerase chain reaction component, and a detection component;

a temperature control device to heat or cool the fluidic channel;

one or more sensors to detect the presence or absence of a magnetic nanoparticle, the one or more sensors adjacent to the temperature control device; and

a processor coupled to the one or more sensors and to the temperature control device.

Clause 9. The apparatus of Clause 8, wherein:

a first reservoir of the one or more reservoirs includes components to extract a nucleotide from cellular material;

a second reservoir of the one or more reservoirs includes PCR reagents to amplify a first nucleotide sequence; and

a third reservoir of the one or more reservoirs includes components to react a second nucleotide sequence with a magnetic nanoparticle (MNP).

Clause 10. The apparatus of any one of Clause 8 or Clause 9, wherein a fourth reservoir of the one or more reservoirs includes components to perform reverse transcription.

Clause 11. The apparatus of any one of Clauses 8-10, wherein at least one sensor of the one or more sensors includes a chemically-bound oligonucleotide probe.

Clause 12. The apparatus of any one of Clauses 8-11, wherein:

a first sensor of the one or more sensors configured to detect a first virus sequence;

a second sensor of the one or more sensors configured to detect a second virus sequence; and

a third sensor of the one or more sensors configured to detect a non-specific virus sequence.

Clause 13. The apparatus of any one of Clauses 8-12, further comprising:

one or more temperature-controlled zones;

one or more chambers for mixing a sample with a reagent;

an element for sensing the presence of a reagent, a fluid, or a combination thereof;

an element for controlling fluid motion; or

a combination thereof.

Clause 14. The apparatus of any one of Clauses 8-13, wherein the element for controlling fluid motion comprises a piezoelectric pump, a coil, a membrane, electrodes, or a combination thereof.

Clause 15. The apparatus of any one of Clauses 8-14, wherein the processor is configured to perform a baseline correction by receiving a plurality of signals and removing at least a portion of the plurality of signals to generate an adjusted signal.

Clause 16. A method for detecting an analyte, comprising:

introducing a sample to an apparatus, the apparatus comprising:
a temperature control device configured to perform a polymerase chain reaction operation; and
one or more sensors to detect the presence or absence of a magnetic nanoparticle;

performing one or more processes to the sample; and

detecting the analyte.

Clause 17. The method of Clause 16, wherein the one or more processes comprise performing a polymerase chain reaction.

Clause 18. The method of any one of Clause 16 or Clause 17, wherein the one or more processes comprise reacting, under reaction conditions, a functionalized MNP and a nucleotide.

Clause 19. The method of any one of Clauses 16-18, wherein the one or more processes comprise:

pretreating the sample;

extracting a nucleotide from cellular material; or

a combination thereof.

Clause 20. The method of any one of Clauses 16-19, wherein the one or more processes comprise performing reverse transcription.

Clause 21. The method of any one of Clauses 16-20, wherein the one or more processes comprise performing a baseline correction on a readout signal.

Clause 22. The method of any one of Clauses 16-21, further comprising detecting a non-specific analyte.

As described herein, the apparatus and methods integrate sample preparation, PCR, and magnetic detection into a single device. The device can be used to detect components of interest in a sample. The apparatus and methods provided herein address major deficiencies of current diagnostic tools and methods by being able to analyze small amounts of analytes, e.g., DNA/RNA, reduce analysis time, lower costs (e.g., elimination of expensive hardware and optics, less people involved in diagnoses, reduced procedures and time), eliminate fluid transfers which cause contamination, among others.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

For purposes of this present disclosure, and unless otherwise specified, all numerical values within the detailed description and the claims herein are modified by “about” or “approximately” the indicated value, and consider experimental error and variations that would be expected by a person having ordinary skill in the art.

As used herein, the indefinite article “a” or “an” shall mean “at least one” unless specified to the contrary or the context clearly indicates otherwise. For example, aspects comprising “a sensor” include aspects comprising one, two, or more sensors, unless specified to the contrary or the context clearly indicates only one sensor is included.

The terms “over,” “under,” “between,” “on,” and other similar terms as used herein refer to a relative position of one layer with respect to other layers. As such, for example, one layer disposed over or under another layer may be directly in contact with the other layer or may have one or more intervening layers. Moreover, one layer disposed between layers may be directly in contact with the two layers or may have one or more intervening layers. In contrast, a first layer “on” a second layer is in contact with the second layer. The relative position of the terms does not define or limit the layers to a vector space orientation of the layers.

The term “coupled” is used herein to refer to elements that are either directly connected or connected through one or more intervening elements. For example, as explained below, a reservoir (e.g., for containing components that are released into a fluidic channel) can be directly connected to the fluidic channel, or it can be connected to the fluidic channel via intervening elements.

The terms “sense” and “detect” are used interchangeably herein to mean obtain information from a physical stimulus. Sensing and detecting include measuring.

While the foregoing is directed to embodiments of the present disclosure, other and further embodiments of the disclosure may be devised without departing from the basic scope thereof, and the scope thereof is determined by the claims that follow.

What is claimed is:

1. An apparatus for detecting a nucleotide sequence, comprising:
a fluidic channel coupled to one or more reservoirs, the fluidic channel comprising a sample introduction component, a polymerase chain reaction component, and a detection component;
a temperature control device to heat or cool the fluidic channel;
one or more sensors to detect the presence or absence of a magnetic nanoparticle; and
a processor coupled to the one or more sensors and to the temperature control device.

2. The apparatus of claim 1, wherein:
a first reservoir of the one or more reservoirs includes components to extract a nucleotide from cellular material;
a second reservoir of the one or more reservoirs includes PCR reagents to amplify a first nucleotide sequence; and
a third reservoir of the one or more reservoirs includes components to react a second nucleotide sequence with a magnetic nanoparticle (MNP).

3. The apparatus of claim 1, wherein a fourth reservoir of the one or more reservoirs includes components to perform reverse transcription.

4. The apparatus of claim 1, wherein at least one sensor of the one or more sensors includes a chemically-bound oligonucleotide probe.

5. The apparatus of claim 1, wherein:
a first sensor of the one or more sensors configured to detect a first virus sequence;
a second sensor of the one or more sensors configured to detect a second virus sequence; and
a third sensor of the one or more sensors configured to detect a non-specific virus sequence.

6. The apparatus of claim 1, further comprising:
one or more temperature-controlled zones;
one or more chambers for mixing a sample with a reagent;
an element for sensing the presence of a reagent, a fluid, or a combination thereof;
an element for controlling fluid motion; or
a combination thereof.

7. The apparatus of claim 1, wherein the processor is configured to perform a baseline correction by receiving a plurality of signals and removing at least a portion of the plurality of signals to generate an adjusted signal.

8. An apparatus for detecting a nucleotide sequence, comprising:
a fluidic channel coupled to one or more reservoirs, the fluidic channel comprising a sample introduction component, a polymerase chain reaction component, and a detection component;
a temperature control device to heat or cool the fluidic channel;
one or more sensors to detect the presence or absence of a magnetic nanoparticle, the one or more sensors adjacent to the temperature control device; and
a processor coupled to the one or more sensors and to the temperature control device.

9. The apparatus of claim 8, wherein:
a first reservoir of the one or more reservoirs includes components to extract a nucleotide from cellular material;
a second reservoir of the one or more reservoirs includes PCR reagents to amplify a first nucleotide sequence; and
a third reservoir of the one or more reservoirs includes components to react a second nucleotide sequence with a magnetic nanoparticle (MNP).

10. The apparatus of claim 8, wherein a fourth reservoir of the one or more reservoirs includes components to perform reverse transcription.

11. The apparatus of claim 8, wherein at least one sensor of the one or more sensors includes a chemically-bound oligonucleotide probe.

12. The apparatus of claim 8, wherein:
a first sensor of the one or more sensors configured to detect a first virus sequence;
a second sensor of the one or more sensors configured to detect a second virus sequence; and
a third sensor of the one or more sensors configured to detect a non-specific virus sequence.

13. The apparatus of claim 8, further comprising:
one or more temperature-controlled zones;
one or more chambers for mixing a sample with a reagent;
an element for sensing the presence of a reagent, a fluid, or a combination thereof;
an element for controlling fluid motion; or
a combination thereof.

14. The apparatus of claim 8, wherein the processor is configured to perform a baseline correction by receiving a plurality of signals and removing at least a portion of the plurality of signals to generate an adjusted signal.

15. A method for detecting an analyte, comprising:
introducing a sample to an apparatus, the apparatus comprising:
a temperature control device configured to perform a polymerase chain reaction operation; and
one or more sensors to detect the presence or absence of a magnetic nanoparticle;
performing one or more processes to the sample; and
detecting the analyte.

16. The method of claim 15, wherein the one or more processes comprise:
performing a polymerase chain reaction;
reacting, under reaction conditions, a functionalized MNP and a nucleotide; or
a combination thereof.

17. The method of claim 15, wherein the one or more processes comprise:
pretreating the sample;
extracting a nucleotide from cellular material; or
a combination thereof.

18. The method of claim 15, wherein the one or more processes comprise performing reverse transcription.

19. The method of claim 15, wherein the one or more processes comprise performing a baseline correction on a readout signal.

20. The method of claim 15, further comprising detecting a non-specific analyte.
